(19) United States
(12) Patent Application Publication
(10) Pub. No.: US 2025/0236605 A1
(43) Pub. Date: Jul. 24, 2025

(54) ALKYL PICOLYL LIGANDS AND USE THEREOF IN ALKOXYCARBONYLATION

(71) Applicant: Evonik Oxeno GmbH & Co. KG, Marl (DE)

(72) Inventors: Stefan Müller, Recklinghausen (DE); Peter Kucmierczyk, Herne (DE); Dirk Fridag, Haltern am See (DE); Julia Büker, Bochum (DE); Ana Markovic, Haltern am See (DE); Roland Peschke, Duisburg (DE); Vincent Bürk, Recklinghausen (DE); Maximilian Wessner, Dortmund (DE); Marius Bilke, Datteln (DE); Robert Franke, Marl (DE)

(73) Assignee: Evonik Oxeno GmbH & Co. KG, Marl (DE)

(21) Appl. No.: 19/029,825

(22) Filed: Jan. 17, 2025

(30) Foreign Application Priority Data
Jan. 22, 2024 (EP) 24153127.6

(51) Int. Cl. C07D 401/12; B01J 31/12; C07C 67/38

(52) U.S. Cl. CPC C07D 401/12; B01J 31/122; C07C 67/38; B01J 2231/321

(57) ABSTRACT

Alkyl picolyl ligands and use thereof in alkoxycarbonylation.

The invention relates to alkyl picolyl ligands and the use thereof in alkoxycarbonylation.

The alkoxycarbonylation of ethylenically unsaturated compounds is a process of increasing significance. An alkoxycarbonylation is understood to mean the reaction of ethylenically unsaturated compounds (olefins) with carbon monoxide and alcohols in the presence of a metal-ligand complex to give the corresponding esters. Typically, the metal used is palladium. The following scheme shows the general reaction equation for an alkoxycarbonylation:

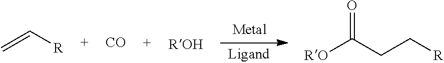

The technical object of the invention is to provide a novel ligand/a novel process which makes it possible to increase the yield of ester.

The object is achieved by a compound according to Claim 1.

Compound having the structure (1) or (2):

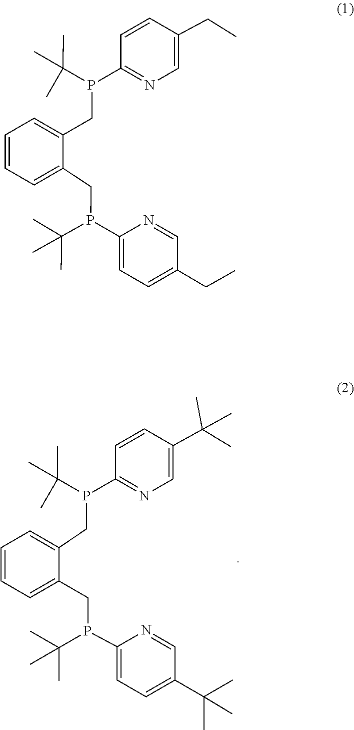 [SMILES: CCc1ccc(P(Cc2ccccc2CP(c2ccc(CC)cn2)C(C)(C)C)C(C)(C)C)nc1 ; CC(C)(C)c1ccc(P(Cc2ccccc2CP(c2ccc(C(C)(C)C)cn2)C(C)(C)C)C(C)(C)C)nc1]

In one embodiment, the compound has the structure (1):

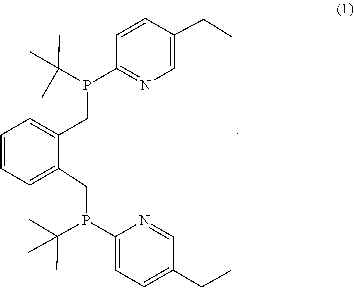 [SMILES: CCc1ccc(P(Cc2ccccc2CP(c2ccc(CC)cn2)C(C)(C)C)C(C)(C)C)nc1]

In one embodiment, the compound has the structure (2):

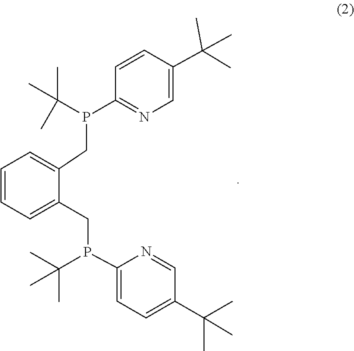 [SMILES: CC(C)(C)c1ccc(P(Cc2ccccc2CP(c2ccc(C(C)(C)C)cn2)C(C)(C)C)C(C)(C)C)nc1]

Also claimed, in addition to the compounds per se, is a process employing the compounds.

Process comprising the process steps of:
a) initially charging an ethylenically unsaturated compound or a mixture of ethylenically unsaturated compounds;
b) adding a compound having the structure (1) or (2):

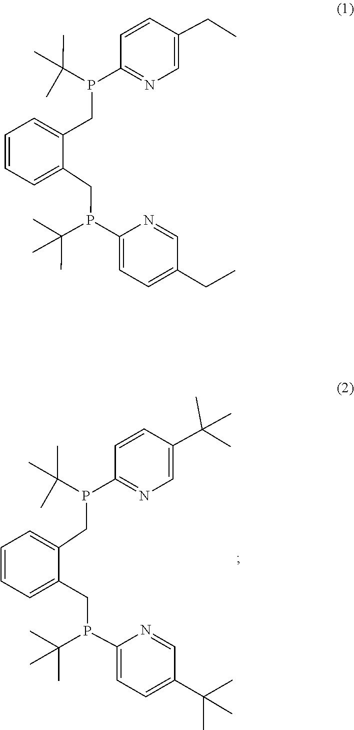 [SMILES: CCc1ccc(P(Cc2ccccc2CP(c2ccc(CC)cn2)C(C)(C)C)C(C)(C)C)nc1 ; CC(C)(C)c1ccc(P(Cc2ccccc2CP(c2ccc(C(C)(C)C)cn2)C(C)(C)C)C(C)(C)C)nc1]

c) adding a Pd compound;
d) adding a co-catalyst selected from: aluminium triflate, H₂SO₄, MSA, pTSA, TFA;
e) adding an alcohol;
f) supplying CO;
g) heating the reaction mixture from a) to f) to convert the ethylenically unsaturated compound to an ester.

In one variant of the process, the Pd compound is selected from: palladium dichloride, palladium(II) acetylacetonate, palladium(II) acetate, dichloro(1,5-cyclooctadiene)palladium(II), bis(dibenzylideneacetone)palladium, bis(acetonitrile)dichloropalladium(II), palladium(cinnamyl)dichloride.

In one variant of the process, the Pd compound is Pd(acac)₂.

In one variant of the process, the co-catalyst is aluminium triflate.

In one variant of the process, the alcohol in process step e) is selected from: methanol, ethanol, 1-propanol, 1-butanol, 1-pentanol, 1-hexanol, 2-propanol, tert-butanol, 3-pentanol, cyclohexanol, phenol, or mixtures thereof.

In one variant of the process, the alcohol in process step e) is methanol.

In one variant of the process, process step f) comprises supplying CO at a pressure in the range from 1 to 5 MPa (10 to 50 bar).

In one variant of the process, process step f) comprises supplying CO at a pressure in the range from 1 to 3 MPa (10 to 30 bar).

In one variant of the process, process step g) comprises heating the reaction mixture to a temperature in the range from 80° C. to 160° C.

In one variant of the process, process step g) comprises heating the reaction mixture to a temperature in the range from 100° C. to 140° C.

In one variant of the process, process step a) comprises initially charging a mixture of ethylenically unsaturated compounds.

In one variant of the process, process step a) comprises initially charging a mixture of n-octenes.

The invention shall now be more particularly elucidated with reference to exemplary embodiments.

The reaction is performed in 20 mL glass vessels with magnetic stirrer bars. Initially Al(OTf)₃ (9.5 mg, 0.2 mol %) and the ligand (0.1 mol %) are weighed into the glass vessel and then airtightly sealed using a crimped septum. A cannula pierced therethrough, which is connected to an argon distributor, ensures an argon atmosphere in the steps which follow and at the same time allows for pressure equalization. The required amount of precursor stock solution (0.8 ml) is added by means of a μl syringe, thus resulting in an addition of Pd(acac)₂ (0.8 mg, 0.025 mol %). This is followed by addition of 0.3 ml of ethylbenzene as internal standard. Methanol is then added using a μl syringe up to a total volume of 8.4 ml. The autoclave is sealed and purged three times with nitrogen. CO is then supplied at a pressure of 20 bar. The reaction solution is then heated to the required temperature of 120° C. The substrate (an n-octene mixture) is added. A sample is taken after 15 minutes.

The employed n-octene mixture consisted of the C8 isomers: 1-octene, cis-2-octene, trans-2-octene, cis-3-octene, trans-3-octene, cis-4-octene and trans-4-octene.

The reaction was performed with the ligands (1), (2) and (3). The experiment with ligand (3) is a comparative experiment.

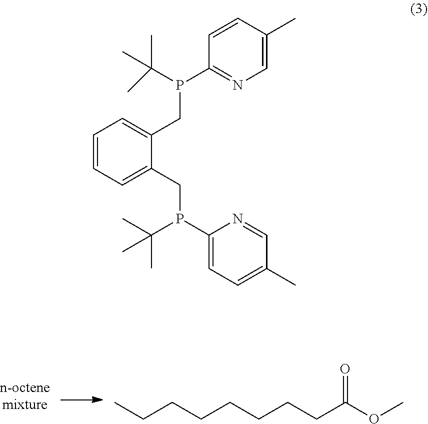

### Reaction Conditions:

Pd(acac)₂, ligand (X), Al(OTf)₃, MeOH, CO: 20 bar, 120° C., 15 min.

The yields of ester are reported in the following table:

| Ligand | Yield |
|---|---|
| (1)* | 62% |
| (2)* | 57% |
| (3) | 55% |

*inventive exemplary embodiment

The experiments performed demonstrate that the stated object is achieved by a compound according to the invention.

1. Compound having the structure (1) or (2):

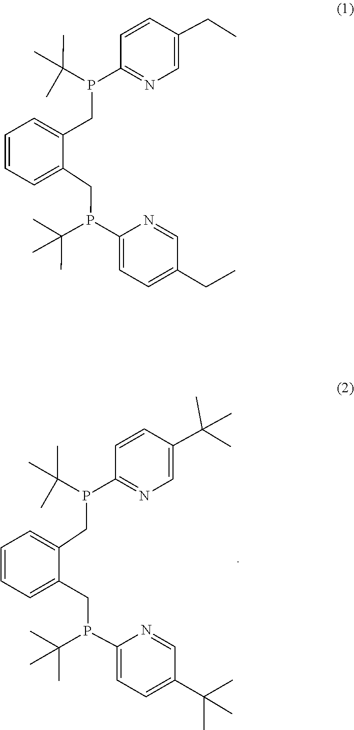 [SMILES: CCc1ccc(P(Cc2ccccc2CP(c2ccc(CC)cn2)C(C)(C)C)C(C)(C)C)nc1 ; CC(C)(C)c1ccc(P(Cc2ccccc2CP(c2ccc(C(C)(C)C)cn2)C(C)(C)C)C(C)(C)C)nc1]

2. Compound according to claim 1,
having the structure (1):

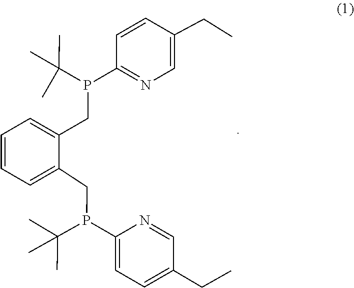 [SMILES: CCc1ccc(P(Cc2ccccc2CP(c2ccc(CC)cn2)C(C)(C)C)C(C)(C)C)nc1]

3. Compound according to claim 1,
having the structure (2):

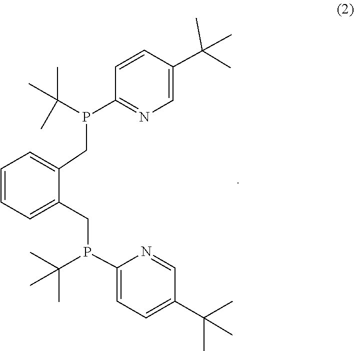 [SMILES: CC(C)(C)c1ccc(P(Cc2ccccc2CP(c2ccc(C(C)(C)C)cn2)C(C)(C)C)C(C)(C)C)nc1]

4. Process comprising the process steps of:
a) initially charging an ethylenically unsaturated compound or a mixture of ethylenically unsaturated compounds;
b) adding a compound having the structure (1) or (2): 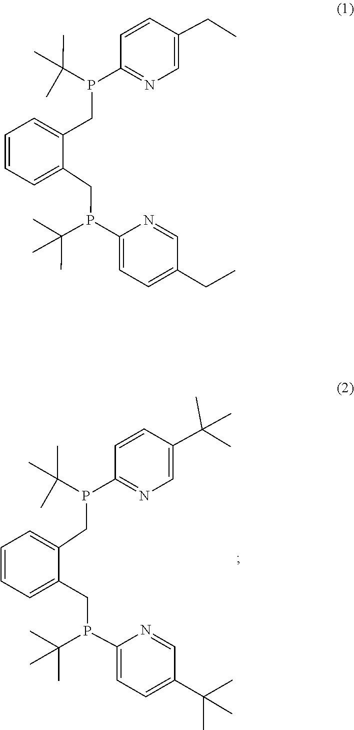 [SMILES: CCc1ccc(P(Cc2ccccc2CP(c2ccc(CC)cn2)C(C)(C)C)C(C)(C)C)nc1 ; CC(C)(C)c1ccc(P(Cc2ccccc2CP(c2ccc(C(C)(C)C)cn2)C(C)(C)C)C(C)(C)C)nc1]
c) adding a Pd compound;
d) adding a co-catalyst selected from: aluminium triflate, H₂SO₄, MSA, pTSA, TFA;
e) adding an alcohol;
f) supplying CO;
g) heating the reaction mixture from a) to f) to convert the ethylenically unsaturated compound to an ester.

5. Process according to claim 4,
wherein the Pd compound is selected from: palladium dichloride, palladium(II) acetylacetonate, palladium(II) acetate, dichloro(1,5-cyclooctadiene)palladium(II), bis(dibenzylideneacetone)palladium, bis(acetonitrile)dichloropalladium(II), palladium(cinnamyl) dichloride.

6. Process according to claim 4,
wherein the Pd compound is Pd(acac)₂.

7. Process according to claim 4,
wherein the co-catalyst is aluminium triflate.

8. Process according to claim 4,
wherein the alcohol in process step e) is selected from: methanol, ethanol, 1-propanol, 1-butanol, 1-pentanol, 1-hexanol, 2-propanol, tert-butanol, 3-pentanol, cyclohexanol, phenol, or mixtures thereof.

9. Process according to claim 4,
wherein the alcohol in process step e) is methanol.

10. Process according to claim 4,
wherein process step f) comprises supplying CO at a pressure in the range from 1 to 5 MPa (10 to 50 bar).

11. Process according to claim 4,
wherein process step g) comprises heating the reaction mixture to a temperature in the range from 80° C. to 160° C.

12. Process according to claim 4,
wherein process step a) comprises initially charging a mixture of ethylenically unsaturated compounds.

13. Process according to claim 4,
wherein process step a) comprises initially charging a mixture of n-octenes.
